(19) United States
(12) Patent Application Publication
(10) Pub. No.: US 2020/0352837 A1
(43) Pub. Date: Nov. 12, 2020

(54) PROCESS FOR PERMANENT WAVING KERATIN FIBERS

(71) Applicant: KAO GERMANY GMBH, Darmstadt (DE)

(72) Inventors: Jörg SCHNEIDER, Darmstadt (DE); Jens HEILMANN, Darmstadt (DE)

(73) Assignee: KAO GERMANY GMBH, Darmstadt (DE)

(21) Appl. No.: 16/764,461

(22) PCT Filed: Nov. 14, 2018
(86) PCT No.: PCT/EP2018/081170

§ 371 Date: May 15, 2020

(30) Foreign Application Priority Data
Nov. 17, 2017 (EP) 17202293.1

(51) Int. Cl. A61K 8/41; A61K 8/22; A61K 8/19; A61Q 5/04

(52) U.S. Cl. CPC A61K 8/416; A61K 8/22; A61K 2800/48; A61Q 5/04; A61K 8/19

(57) ABSTRACT

Present invention relates to a process for permanent waving keratin fibers, especially human hair, for achieving durable waves wherein fibers are treated with a reducing composition, a non-reducing and non-oxidizing alkaline composition and finally an oxidizing composition wherein after processing the reducing composition the fibers are rinsed off.

Present invention relates to a process for permanent waving keratin fibers, especially human hair, for achieving durable waves.

The well-known and commonly used process for permanent waving keratin fibers involves reducing and oxidizing steps which is often perceived as fiber damaging and the results are very much dependent upon how the whole process is carried out. The fiber damage is especially due to the inappropriately selected, if not adjusted, processing period of the reducing composition. The hair may easily become over processed and appear, therefore, to have less strength, be brittle and especially not naturally feeling upon touching.

Another aspect is that the use and/or the need of heat application during processing of the reducing agent on the fibers. This is usually realized with an external electrical heating devices and, especially in Asian geography, a specially therefore designed machine so called heat perming machine is used. It is the observation of the applicant that unless the pre-reduced hair is processed with heat, almost no curls are obtained although the hair is considerably damaged. There is highly need for simplified and non-damaging processes for obtaining strong, natural feeling, homogenous permanent waving.

The EP 673 640 discloses a process for permanent waving hair wherein hair is treated with a reducing composition for a period of 20 min and, without rinsing off, an alkaline composition was applied comprising alkali carbonates and hydrogen carbonates for a period of 10 min and finally hair is applied an aqueous oxidizing composition. It has been observed that the process described therein does not deliver cosmetically acceptable hair qualities in terms of waving efficiency and especially in smoothness and softness.

Furthermore, JP-H03-153621 discloses a permanent shaping process wherein an acidic composition is mixed with an alkaline reducing agent composition and applied onto hair and after certain processing time an oxidizing composition is applied onto hair. The process does not deliver cosmetically appealing curls and hair qualities.

Similar process to the above is disclosed in US2008/0142033 wherein after treating hair with reducing composition, a composition comprising monovalent cation salt of organic acids is applied and finally hair is oxidized. This process as well have drawbacks in delivering less damage and effective curling to the hair which at the same time feels soft and smooth upon touching.

The inventors of the present invention has unexpectedly found out that application of an intermediate aqueous alkaline composition onto reduced keratin fibers, especially human hair, wherein the reducing composition is rinsed off delivers soft and smooth hair with well-defined strong curls. The hair waved with such processes feels natural upon touching, has natural appearance with homogenous and intensive bouncy curls.

Thus, the first object of the present invention is a process for permanent waving keratin fibers, especially human hair, wherein,
a—optionally, the keratin fibers especially human hair is washed and/or shampooed, and towel dried,
b—an aqueous composition comprising one or more reducing agent is applied and left on the fibers for a period of 1 to 60 min,
c—the fibers are rinsed off,
d—the fibers are put on curlers,
e—a non-reducing and non-oxidizing aqueous composition comprising one or more alkalizing agent is applied onto fibers and left on the fibers for a period 1 to 60 min,
f—optionally the fibers are rinsed off,
g—an aqueous composition comprising one or more oxidizing agents, preferably hydrogen peroxide or bromate salt, is applied onto fibers and left on the fibers for a period 1 to 30 min,
h—the fibers are optionally rinsed off, and
i—the fibers are dried,
wherein the curlers are taken off from fibers before or during processing in step g or after the step g prior to rinsing off and/or drying.

The second object of the present invention is the use of a process of the present invention for achieving natural, intensive, homogeneous waves on keratin fibers, especially human hair.

The third object of the present invention is a kit for keratin fibers, especially human hair comprising the compositions used in the process above, namely an aqueous composition comprising one or more reducing agents, a non-reducing and non-oxidizing aqueous composition comprising one or more alkalizing agents, and an aqueous composition comprising one or more oxidizing agents, preferably hydrogen peroxide or bromate salt.

In a further preferred embodiment of the present invention, in order to prevent fibers drying during processing, the fibers, especially human hair, are covered with e.g. foil or towel, especially during the periods the compositions comprising reducing agents and alkalizing agents are left on the fibers.

In a further preferred embodiment of the present invention, the whole process is carried out at ambient temperature without using any heat and/or heating device. Without being bound by the theory, this should even further be beneficial to reduce hair damage and therefore contribute to healthy appearance and feeling of the fibers.

In the process of the present invention, an aqueous composition comprising one or more reducing agents is applied onto fibers. In principal any reducing agent of inorganic and organic ones and their mixtures are suitable for the purpose of the present invention. The preferred ones are inorganic and organic reducing agents.

Suitable inorganic reducing agents are sulfite and/or hydrogen sulfite salts such as sodium, potassium, ammonium and suitable organic reducing agents are thiogylcolic acid and/or its salts, cysteamine and/or its salts, thioglycerin and/or its salts, glycerin esters of thioglycolic acid and/or its salts, thiolactic acid and/or its salts, cysteine or its derivatives and/or its salts, and their mixtures. Preferred are thioglycolic acid and/or its salts, thiolactic acid and/or its salts, cysteine or its derivatives and/or its salts and sodium, potassium, ammonium sulfites and their mixtures. The most preferred are thioglycolic acid and/or its salts and sodium, potassium, ammonium sulfites, and their mixtures.

The total concentration of reducing agents in the aqueous composition of step b is in the range of 0.5 to 20%, preferably 1 to 15%, more preferably 2 to 12% and most preferably 3 to 10% by weight calculated to the total of the aqueous composition.

The pH of the composition may be acidic or alkaline and preferably in the range of 3 to 12, more preferably 4 to 11 and most preferably it is alkaline and in the range of 7.5 to 10.5. The pH may be adjusted with the known organic and/or inorganic acids and alkalizing agents (see below).

The aqueous composition comprising one or more reducing agents is left on the hair for a period of 1 to 60 min, preferably 2 to 45 min, more preferably 5 to 30 min and most preferably 5 to 20 min at ambient temperature and without using any heat and/or heating device.

After rinsing off the fibers, the fibers are put on curlers and applied a non-reducing and non-oxidizing aqueous composition comprising one or more alkalizing agents. The composition is left on the hair for a period of 1 to 60 min, preferably 2 to 45 min, more preferably 5 to 30 min and most preferably 5 to 20 min at ambient temperature and without using any heat and/or heating device.

The suitable alkalizing agents may be inorganic and organic ones and all well-known agents are suitable for the purpose of the present process. The pH of the composition is in the range of 7.5 to 12, preferably 8 to 11, more preferably 8.5 to 10.5 and most preferably 8.5 to 10. The pH may be adjusted by selecting the concentration of the alkalizing agent for achieving the required pH or alternatively may be adjusted using inorganic and/or organic acids.

The concentration of the alkalizing agents is adjusted as their NH₃ equivalent which may easily be determined by titrating the alkaline solution with a standard acidic solution and calculating the molar concentration of the alkalizing agent which is then expressed as molar equivalent NH₃. Finally from the molar equivalent and from the molecular weight of NH₃, the concentration in % is calculated. The titration is carried out at ambient temperature i.e. at approximately 20° C. Accordingly, the ammonia equivalent concentration must be in the range of 0.1 to 15%, preferably 0.1 to 12.5%, more preferably 0.25 to 10% and most preferably 0.5 to 8% by weight calculated to the total of the composition as ammonia equivalent determined by titration method at ambient temperature.

Suitable alkalizing agents are the alkali hydroxides such as sodium hydroxide, potassium hydroxide, ammonia and its salts such as ammonium carbonate, ammonium hydrogen carbonate, ammonium carbamate, ammonium chloride, ammonium sulfate, ammonium phosphates such as ammonium dihydrogen phopshate, diammonium hydrogen phosphate, diammonium sodium phosphate, ammonium sodium hydrogen phosphate or ammonium disodium phosphate, ammonium nitrate, ammonium bromide, ammonium iodide, ammonium thiosulfate, ammonium molybdate, ammonium vanadate, ammonium sulfamate, ammonium citrate, ammonium salicylate, ammonium valerate, ammonium tartarate, ammonium benzoate, ammonium acetate, ammonium formiate and ammonium lactate, guanidine and its salts such as guanidine hydrochloride, guanidine carbonate, guanidine bicarbonate, and an alkyl or alkanol amine according to the general structure

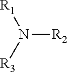

wherein R₁, R₂, and R₃ are same or different H, from C₁ to C₄, C₃ to C₄ unsaturated alkyl, C₃ to C₄ branched alkyl, C₁ to C₄ hydroxyl alkyl, C₃ to C₄ unsaturated hydroxyl alkyl, C₃ to C₄ branched hydroxyl alkyl, with the condition that at least one of R₁, R₂, or R₃ is different from H, such as monoethanolamine, diethanolamine, triethanolamine, monoethanol methylamine, monoethanoldimethylamine, di-ethanolmethylamine, monoethanolethylamine, monoethanoldiethylamine, diethanolethylamine, monoethanolpropylamine, monoethanoldipropylamine, diethanolpropylamine, monoethanolbutylamine, diethanolbutylamine and amino methyl propanol and their mixtures.

Preferred are ammonia and its salts, monoethanolamine, diethanolamine, triethanolamine, amino methyl propanol, guanidine salts and their mixtures. The most preferred are ammonia, ammonium chloride, guanidine carbonate, monoethanolamine and amino methyl propanol and their mixtures.

After rinsing off the aqueous composition comprising alkalizing agents, an aqueous composition comprising one or more oxidizing agents, preferably hydrogen peroxide or a bromate salt is applied onto hair and left on the hair for 0.5 to 30 min, preferably 2 to 25 min, more preferably 3 to 20 min and most preferably 5 to 15 min at ambient temperature without application of any heat and/or heating device.

The fibers are preferably rinsed off at the end of the above referred processing time. Optionally the oxidizing composition may also be left on the hair, i.e. not rinsed off from hair.

The total concentration of one or more oxidizing agents, preferably hydrogen peroxide or bromate salt, in the aqueous composition is in the range of 0.1 to 15%, preferably 0.2 to 12.5%, more preferably 0.25 to 10% and most preferably 0.5 to 8% by weight calculated to the total of the aqueous composition.

In general the pH of the oxidizing composition is in the range of 2 to 8. The pH of the composition is depending on the oxidizing agent comprised in the composition. In case of hydrogen peroxide a pH in the range of 2 to 6 is suitable. In case of sodium bromate a pH of 5 to 8 is suitable. pH of the composition may be adjusted using inorganic and/or organic acids and bases well known in the art.

The curlers are being taken off from hair prior to application of the aqueous oxidizing composition or during the time the aqueous composition is left on the hair or after rinsing off the aqueous oxidizing composition. The preferred is the curlers are taken off from hair after rinsing off the aqueous oxidizing composition.

In case that the aqueous oxidizing composition is not rinsed off from hair, the curlers may be taken off from hair either after application of the oxidizing composition or prior to application of the oxidizing composition.

In the following, all reported concentrations must be understood as relative to each of the compositions because, firstly, the compositions are not mixed with each other and secondly, the same ingredient disclosed must not be comprised in all of the composition, although this is a possibility.

Aqueous compositions, all three or one or two advantageously comprise a thickening agent, preferably a thickening polymer. Suitable and preferred ones are thickening polymers such as polysaccharides such as alginate, pectinate, xanthan, hydroxypropyl xanthan or dehydroxanthan, non-ionic polysaccharides such as cellulose ethers (e.g., methylcellulose, hydroxyethylcellulose (HEC), methyl hydroxyethylcellulose (MHEC), ethyl hydroxyethylcellulose (EHEC), methyl ethyl hydroxyethylcellulose (MEHEC)), starch or dextrins. Synthetic acrylate type of thickeners may as well be comprised such as acrylate copolymers and alkyl acrylates homo or copolymers also known as associative thickeners.

The concentration of the thickening polymer is very much dependent on the type of the thickening polymer and the targeted consistency (viscosity) of the compositions. Typically, the thickening polymers are comprised in the compositions at a concentration in the range of 0.1 to 3%, preferably 0.25 to 2% by weight calculated to the total of each of the composition.

Aqueous compositions, all three or one or two can comprise one or more fatty alcohols. Suitable fatty alcohols are the ones with the chain length of 14 to 22 C atoms which may be saturated or unsaturated, linear or branched which may as well be substituted. Non-limiting examples are myristyl alcohol, cetyl alcohol, stearyl alcohol, behenyl alcohol, octyl dodecanol, cetostearyl alcohol, and their mixtures.

The total concentration of fatty alcohol is in the range from 0.5 to 15%, preferably 1 to 10% by weight, calculated to total of each of the composition.

Aqueous compositions, all three or one or two, advantageously comprise one or more surfactants. Suitable ones are selected from anionic, non-ionic, amphoteric and cationic ones.

Anionic surfactants suitable are in principle known from the cleansing compositions. These are anionic surfactants of the sulfate, sulfonate, carboxylate and alkyl phosphate type, for example, the known C₁₀-C₁₈-alkyl sulfates, and in particular the respective ether sulfates, for example, C₁₂-C₁₄-alkyl ether sulfate, lauryl ether sulfate, especially with 1 to 4 ethylene oxide groups in the molecule, monoglyceride (ether) sulfates, fatty acid amide sulfates obtained by ethoxylation and subsequent sulfatation of fatty acid alkanolamides, and the alkali salts thereof, as well as the salts of long-chain mono- and dialkyl phosphates. Preferred anionic surfactants are alkyl sulphate surfactants especially lauryl sulphate and its salts.

Further suitable surfactants are nonionic surfactants. Non-limiting examples are long-chain fatty acid mono- and dialkanolamides, such as coco fatty acid mono- or diethanolamide and myristic fatty acid mono or diethanolamide, stearic acid mono or diethanolamide, alkyl polyglucosides with an alkyl group of 8 to 18 carbon atoms, and with 1 to 5 glucoside units, sorbitan esters, such as polyethylene glycol sorbitan stearic, palmitic, myristic and lauric acid esters, fatty acid polyglycol esters or poly-condensates of ethyleneoxide and propyleneoxide, as they are on the market, for example, under the trade name “Pluronics®”, as well as fatty alcohol ethoxylates, C₁₀-C₂₂-fatty alcohol ethoxylates, known by the generic terms “Laureth”, “Myristeth”, “Oleth”, “Ceteth”, “Deceth”, “Steareth” and “Ceteareth” according to the CTFA nomenclature, including addition of the number of ethylene oxide molecules, e.g., “Laureth-16”: The average degree of ethoxylation thereby ranges between about 2.5 and about 100, preferably about 10 and about 30.

Suitable amphoteric surfactants are in particular the various known betaines such as alkyl betaines, fatty acid amidoalkyl betaines and sulfobetaines, for example, lauryl hydroxysulfobetaine; long-chain alkyl amino acids, such as cocoaminoacetate, cocoaminopropionate and sodium cocoamphopropionate and -acetate have also been proven suitable.

Suitable cationic surfactants are according to the general structure

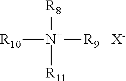

wherein R₈ is a saturated or unsaturated, branched or linear alkyl chain with 8-22 C atoms or

R₁₂CONH(CH₂)ₙ

where R₁₂ is saturated or unsaturated, branched or linear alkyl chain with 7-21 C atoms and n has typical value of 1-4 or

R₁₃COO(CH₂)ₙ

where R₁₃ is saturated or unsaturated, branched or linear alkyl chain with 7-21 C atoms and n has typical value of 1-4, and

R₉ is H or unsaturated or saturated, branched or linear alkyl chain with 1-22 C atoms or

R₁₂CONH(CH₂)ₙ

or

R₁₃COO(CH₂)ₙ

where R₁₂, R₁₃ and n are same as above.

R₁₀ and R₁₁ are H or lower alkyl chain with 1 to 4 Carbon atoms, and X is typically chloride, bromide, methosulfate.

Typical examples of those ingredients are cetyl trimethyl ammonium chloride, stearyl trimonium chloride, dipalmitoyl dimonium chloride, distearyl dimethyl ammonium chloride, stearamidopropyl trimonuim chloride, dioleoylethyl dimethyl ammonium methosulfate, dioleoylethyl hydroxyethylmonium methosulfate.

The total concentration of one or more surfactants is in the range of 0.1 to 12.5%, preferably 0.2 to 10% and more preferably 0.5-7.5% by weight, calculated to the total of each of the composition.

Further advantageously, aqueous compositions, all three or one or two, comprise one or more silicone compound, preferably silicone oil. Suitable and preferred ones are known with their CTFA adopted name as dimethicone and commercially available from Dow Corning under the trade name DC 200 with various viscosities.

Further advantageously, aqueous compositions, all three or one or two, comprise one or more cationic polymers as conditioning and/or thickening agents. Those are cationic cellulose type polymers know as Polymer JR type from Amerchol such as Polyquaternium 10 or cationic guar gum known with trade name Jaguar from Rhone-Poulenc and chemically for example Guar hydroxypropyl trimonium chloride. Furthermore, chitosan and chitin can also be included in the compositions as cationic natural polymers.

Furthermore, it has been found suitable those cationic polymers known with their CTFA category name Polyquaternium. Typical examples of those Polyquaternium 4, Polyquaternium 6, Polyquaternium 7, Polyquaternium 10, Polyquaternium 11, Polyquaternium 16, Polyquaternium 22, Polyquaternium 24, Polyquaternium 28, Polyquaternium 30, Polyquaternium 37, Polyquaternium 36, Polyquaternium 46, Polyquaternium 67, and Polyquaternium 72.

Preferred are Polyquaternium-2, Polyquaternium-6 and Polyquaternium 16.

The total concentration of cationic polymers may be in the range of 0.1-2.5%, preferably 0.25-2% by weight and more preferably 0.5-1.5% by weight, calculated to total of each of the composition.

Further advantageously, aqueous compositions all three or one or two comprise one or more aminated silicones which may be selected from amodimethicones and grafted aminianted siliocnes. Suitable ones are available under various trade names such as DC 969, Belsil from Wacker Chemie AG and know with the CTFA adopted name Amodimethicone, and Elastomer OS from Kao Corporation known with CTFA adopted name Polysilicone-9.

Furthermore, aqueous compositions, all three or one or two, comprise one or more organic solvent which may act as penetration enhancer and/or solubilizing agent for the compounds not readily soluble in the aqueous medium. The suitable ones are 2-phenoxyethanol, benzyl alcohol, 2-phenylethanol and 2-benzyloxyethanol. Suitable aliphatic alcohols are ethanol, isopropanol, propanol, n-butanol, isobutanol, t-butanol and 1-pentanol.

Concentration of one or more organic solvent is in the range of 0.1 to 15%, preferably 0.5 to 12.5% and more preferably 1 to 10% and most preferably 1 to 7.5% by weight calculated to the total of each of the composition.

The aqueous compositions, all three or one or two may advantageously comprise urea, at a concentration in the range of 0.1 to 20%, preferably 1 to 15% by weight calculated to the total of the compositions.

Additionally, the aqueous compositions, all three or one or two, comprise one or more polyols. Suitable ones are glycerine, phytantriol, panthenol, ethyleneglycol, polyethyleneglycols, propylene glycols such as 1,2 propylene glycol, 1,3-propylene glycol and polypropylene glycols.

The total concentration of one or more polyol is in the range of 0.1 to 15%, preferably 0.25 to 12.5%, more preferably 0.5 to 10% and most preferably 1 to 7.5% by weight calculated to the total of each of the composition.

The aqueous compositions, all three or one or two, can comprise one or more amino acids and/or their water soluble salts. Suitable ones are glycine, histidine, citrullin, asaparagine, alanine, valine. Leucine, isoleucine, proline, tryptophan, phenylalanine, methinone, serine, tyrosine, threonine and glutamine.

The total concentration of one or more aminoacids and/or their water soluble salts is in the range of 0.01 to 2.5%, preferably 0.1 to 2%, more preferably 0.15 to 1.5% and most preferably 0.2 to 1% by weight calculated to the total of each of the composition.

Any of the compositions described in detail above may comprise ingredients customarily found in such compositions such as preservative, fragrance, chelating agents, radical scavenger, etc.

Following example to illustrate the invention, but not to limit it.

### EXAMPLE 1

Aqueous Reducing Composition

| | % by weight |
|---|---|
| Ammonium thioglycolate | 10 |
| Ammonium hydroxide | 2 |
| Water | to 100 |

The pH of the above composition was adjusted to pH 9.0.

Aqueous Alkaline Composition

| | % by weight |
|---|---|
| Ammonium hydroxide | 5 |
| Water | to 100 |

The pH of the above composition was adjusted to pH 9.9.

Aqueous Oxidizing Composition

| | |
|---|---|
| Hydrogen peroxide | 3 |
| Phosphoric acid | q.s. to pH 3 |
| Water | to 100 |

A hair streak weighing approximately 5 g and having a length of 20 cm is permanently waved using the above compositions. Firstly the streak was washed with a commercially available shampoo compositions and towel dried. Afterwards, the streak was dipped into the aqueous reducing composition and left in the solution for 15 min and taken out and rinsed off with water. Afterwards, the streak was put on curlers with a diameter of 1.5 cm and an alkaline dipped into the alkaline composition of above. After 10 min, taken out and rinsed off and dipped into the oxidizing composition for 15 min and the curlers were taken off. It was observed that the streak was well waved.

### EXAMPLE 2

Aqueous Reducing Composition

| | % by weight |
|---|---|
| Ammonium thioglycolate | 7 |
| Ammonium bicarbonate | 4 |
| Phosphoric acid | q.s. to pH 6.5 |
| Water | to 100 |

Aqueous Alkaline Composition

| | % by weight |
|---|---|
| Ammonium bicarbonate | 4 |
| Sodium hydroxide | q.s. to pH 8.5 |
| Water | to 100 |

Aqueous Oxidizing Composition

| | % by weight |
|---|---|
| Sodium bromate | 5 |
| Water | to 100 |

The above composition had a pH of 7.0.

Using the above compositions, two hair streaks as in example 1 were permanently waved comparatively to the process disclosed in EP 673640.

The hair streaks were shampooed and the first reducing agents were applied and left on the streaks for 20 min, Afterwards, one of the streaks was rinsed off with water and the other was not rinsed off. Both streaks were put on curlers having a dimeter of 1.5 cm. Both streaks were soaked into the alkaline composition and left for 10 min. Subsequently, the streak not rinsed off was directly soaked into the oxidizing composition whereas the other first rinsed off and afterwards soaked into the oxidizing composition. After 15 min both were taken out from the oxidizing composition and rinsed off with water and the curlers were taken off. The streaks were dried with a hair drier.

It was observed that the streak which was rinsed off after treating with reducing agent was waved much more effectively and also felt smoother and softer upon touching. The streaks were analyzed by a panel of 7 hair dressers for their waving appearance, softness and smoothness in a scale 1 to 5 wherein 1 is not good at all and 5 is being the best performance. The following results were obtained for the streaks treated according to the present invention and comparative process according to EP673640.

| | Inventive | SD | Comparative | SD |
|---|---|---|---|---|
| Wave appearance | 3.50 | 0.40 | 2.45 | 0.38 |
| Softness | 3.71 | 0.45 | 2.52 | 0.44 |
| Smoothness | 3.85 | 0.35 | 2.42 | 0.49 |

The SD means standard deviation of the mean values.

From above results it is beyond any doubt that the process according to the process of the present invention delivers very much improved permanent waved hair. Since there is only one difference which is the rinsing off steps between the applications of various compositions, the improved results are clearly attributed to this.

### EXAMPLE 3

Aqueous Reducing Composition

| | % by weight |
|---|---|
| Ammonium thioglycolate | 7 |
| Ammonium bicarbonate | 4 |
| Phosphoric acid | q.s. to pH 6.5 |
| Water | to 100 |

Aqueous Alkaline Composition

| | % by weight |
|---|---|
| Ammonium bicarbonate | 4 |
| Sodium hydroxide | q.s. to pH 8.5 |
| Water | to 100 |

Aqueous Oxidizing Composition

| | % by weight |
|---|---|
| Sodium bromate | 5 |
| Water | to 100 |

The above composition had a pH of 7.0.

Using the above compositions, two hair streaks as in example 1 were permanently waved comparatively to the process disclosed in EP 673640.

The hair streaks were shampooed and the first reducing agents were applied and left on the streaks for 20 min, Afterwards, one of the streaks was rinsed off with water and the other was not rinsed off. Both streaks were put on curlers having a diameter of 1.5 cm. Both streaks were soaked into the alkaline composition and left for 10 min. Subsequently, both streaks were directly soaked into the oxidizing composition and after 15 min both were taken out from the oxidizing composition and rinsed off with water and the curlers were taken off. The streaks were dried with a hair drier.

It was observed that the streak which was rinsed off after treating with reducing agent was waved much more effectively and also felt smoother and softer upon touching. The streaks were analyzed by a panel of 7 hair dressers for their waving appearance, softness and smoothness in a scale 1 to 5 wherein 1 is not good at all and 5 is being the best performance. The following results were obtained for the streaks treated according to the present invention and comparative process according to EP673640.

| | Inventive | SD | Comparative | SD |
|---|---|---|---|---|
| Wave appearance | 3.65 | 0.41 | 2.23 | 0.31 |
| Softness | 3.77 | 0.43 | 2.41 | 0.38 |
| Smoothness | 3.95 | 0.33 | 2.23 | 0.34 |

The SD means standard deviation of the mean values.

From above results it is beyond any doubt that the process according to the process of the present invention delivers very much improved permanent waved hair. Since there is only one difference which is the rinsing off steps prior to the application of alkaline composition, the improved results are clearly attributed to this difference.

The following compositions are used in the process of the present invention.

### EXAMPLE 4

Aqueous Reducing Composition

| | % by weight |
|---|---|
| Cetearyl alcohol | 10 |
| Oleyl alcohol | 5 |
| Thioglycolic acid | 1.7 |
| Ceteareth-20 | 1.0 |
| Hydroxyethylcellulose | 0.5 |
| EDTA | 0.5 |
| Fragrance | 0.5 |
| Aminomethylpropanol | q.s. to pH 9.0 |
| Water | to 100 |

Aqueous Alkaline Aerosol Foam Composition

| | % by weight |
|---|---|
| Ammonium hydroxide | 5 |
| Ammonium chloride | 2 |
| Monoethanolamine | q.s. 9.0 |
| Cetrimonium chloride | 2 |
| Hydroxyethylcellulose | 0.5 |
| Water | to 100 |

The composition was filled in an aerosol can with 90% the above composition and 10% propane butane mixture as the propellant. The can was equipped with a foam dispensing actuator and head.

Aqueous Oxidizing Composition

| | % by weight |
|---|---|
| Cetearyl alcohol | 5 |
| Behenrimonium chloride | 2 |
| Hydrogen peroxide | 3 |
| Phosphoric acid | to pH 3.0 |
| Water | to 100 |

The above composition was applied in the same way of the inventive process of the previous examples. The hair streak was shampooed and the first reducing agents were applied and left on the streaks for 20 min, Afterwards, the streak was rinsed off with water and was put on curlers having a dimeter of 2.0 cm. The streak was soaked into the alkaline composition and left for 10 min. Subsequently, the streak was rinsed off with water and was soaked into the oxidizing composition and after 15 min. it was taken out from the oxidizing composition and rinsed off with water and the curlers was taken off. The streak was dried with a hair drier. It was observed that the streak was effectively and homogeneously curled and felt soft and smooth upon touching.

1. A process for permanently waving keratin fibers, especially human hair, wherein,
a—optionally, the fibers are washed and/or shampooed, and towel dried,
b—a first aqueous composition comprising one or more reducing agent is applied and left on the fibers for a period of 1 to 60 min,
c—the fibers are rinsed off,
d—the fibers are put on curlers,
e—a non-reducing and non-oxidizing second aqueous composition comprising one or more alkalizing agent is applied onto fibers and left on the fibers for a period 1 to 60 min,
f—optionally the fibers are rinsed off,
g—a third aqueous composition comprising one or more oxidizing agents, is applied onto the fibers and left on the fibers for a period 1 to 30 minutes,
h—the fibers are optionally rinsed off, and
i—the fibers are dried,
wherein the curlers are taken off from fibers before or during processing in step g or after the step g prior to rinsing off and/or drying.

2. The process according to claim 1 wherein the fibers are covered during the period the first and second aqueous compositions are left on the fibers.

3. The process according to claim 1, wherein the reducing agents are selected from sulfite and/or hydrogen sulfite salts, thiogylcolic acid and/or its salts, cysteamine and/or its salts, thioglycerin and/or its salts, glycerin esters of thioglycolic acid and/or its salts, thiolactic acid and/or its salts, cysteine or its derivatives and/or its salts, and their mixtures.

4. The process according to claim 1, wherein the total concentration of the reducing agents in the first aqueous composition of step b is in the range of 0.5 to 20%, by weight, calculated to the total of the aqueous composition.

5. The process according to claim 1, wherein the first aqueous composition has a pH in the range of 3 to 12.

6. The process according to claim 1, wherein the process as a whole is carried out at ambient temperature without using any heat and/or heating device.

7. The process according to claim 1, wherein the pH of the second aqueous composition in step e is in the range of 7.5 to 12.

8. The process according to claim 1, wherein the second aqueous composition comprises alkalizing agent at an ammonia equivalent concentration of 0.1 to 15% by weight calculated to the total of the second aqueous composition as ammonia equivalent determined by titration method.

9. The process according to claim 1, wherein the second aqueous composition comprises one or more alkalizing agents selected from one or more alkali hydroxides chosen from sodium hydroxide and potassium hydroxide; one or more of ammonia and its salts chosen from ammonium carbonate, ammonium hydrogen carbonate, ammonium carbamate, ammonium chloride and ammonium sulfate; one or more ammonium phosphates chosen from ammonium dihydrogen phosphate, diammonium hydrogen phosphate, diammonium sodium phosphate, ammonium sodium hydrogen phosphate and ammonium disodium phosphate; ammonium nitrate, ammonium bromide, ammonium iodide, ammonium thiosulfate, ammonium molybdate, ammonium vanadate, ammonium sulfamate, ammonium citrate, ammonium salicylate, ammonium valerate, ammonium tartarate, ammonium benzoate, ammonium acetate, ammonium formiate and ammonium lactate; one or more of guanidine and its salts chosen from guanidine hydrochloride, guanidine carbonate and guanidine bicarbonate and an alkyl or alkanol amine according to the general structure 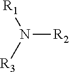
wherein R₁, R₂, and R₃ are same or different H, from C₁ to C₄, C₃ to C₄ unsaturated alkyl, C₃ to C₄ branched alkyl, C₁ to C₄ hydroxyl alkyl, C₃ to C₄ unsaturated hydroxyl alkyl, C₃ to C₄ branched hydroxyl alkyl, with the condition that at least one of R₁, R₂, or R₃ is different from H, and is chosen from monoethanolamine, diethanolamine, triethanolamine, monoethanol methylamine, monoethanoldimethylamine, di-ethanolmethylamine, monoethanolethylamine, monoethanoldiethylamine, diethanolethylamine, monoethanolpropylamine, monoethanoldipropylamine, diethanolpropylamine, monoethanolbutylamine, diethanolbutylamine and amino methyl propanol and their mixtures.

10. The process according to claim 1, wherein the alkalizing agent is selected from ammonia, amino methyl propanol, monoethanolamine, sodium hydroxide, potassium hydroxide, diethanolamine, thriethanolamine, and mixtures thereof.

11. The process according to claim 1, wherein the pH of the third aqueous composition is in the range of 2 to 8 when hydrogen peroxide is comprised as oxidizing agent and in the range of 5 to 8 when bromate salt is comprised in the third aqueous composition.

12. The process according to claim 1, wherein hydrogen peroxide or bromate salt in the third aqueous composition are comprised at a concentration in the range of 0.1 to 10% by weight, calculated to the total of the third aqueous composition.

13. The process according to claim 1, wherein the curlers are taken off from the fibers after rinsing off the third aqueous composition and prior to drying the fibers.

14. The process according to claim 1, wherein at least one of the aqueous compositions comprise a thickening agent being a thickening polymer or one or more surfactants selected from anionic, nonionic, amphoteric and cationic ones.

15. Kit for keratin fibers, especially human hair, comprising
a first aqueous composition comprising one or more reducing agent,
a non-reducing and non-oxidizing second aqueous composition comprising one or more alkalizing agent, and
a third aqueous composition comprising one or more oxidizing agents.

16. The kit according to claim 15, wherein the third aqueous composition is hydrogen peroxide or a bromate salt.
